(19) United States
(12) Patent Application Publication
(10) Pub. No.: US 2015/0013674 A1
(43) Pub. Date: Jan. 15, 2015

(54) SYSTEM AND METHOD FOR MONITORING AND REPORTING STATUS OF A VENTILATED PATIENT

(71) Applicant: Covidien LP, Mansfield, MA (US)

(72) Inventors: Peter Doyle, Vista, CA (US); Gardner James Kimm, Carlsbad, CA (US); Mehdi M. Jafari, Laguna Hills, CA (US); Warren Sanborn, Escondido, CA (US); Sidney A. Jacobi, San Marcos, CA (US); Robert Hartman, San Marcos, CA (US)

(21) Appl. No.: 13/942,019

(22) Filed: Jul. 15, 2013

(51) Int. Cl. A61M 16/00

(52) U.S. Cl. CPC A61M 16/0051; A61M 16/0003; A61M 16/0057; A61M 2016/0042; A61M 2016/0027
USPC 128/202.22

(57) ABSTRACT

The present disclosure provides a method of issuing an alert for optimizing interaction between a patient and a ventilatory system. The method includes ventilating a patient using a ventilatory system and retrieving one or more of current ventilatory settings, collected ventilatory data, and patient data. The method also includes detecting corruption of interaction between a patient and the ventilatory system, determining one or more root causes of the corruption, and determining and displaying a recommendation for resolving the corruption based on the one or more root causes.

### TECHNICAL FIELD

The present disclosure relates generally to the field of medical equipment for respiratory therapy and more specifically to a user interface for a ventilator used for monitoring and controlling the breathing of a patient.

### BACKGROUND

Modern patient ventilators are designed to ventilate a patient's lungs with breathing gas, and to thereby assist a patient when the patient's ability to breathe on his own is somehow impaired. As patient ventilator systems and their various components, including sensors and control systems, have become more sophisticated, and more understanding is gained about the physiology of breathing and the infirmities and damage which form the requirements for respiratory therapy, the number of variables to be controlled and the timing and interrelationships between the parameters provide for a complex environment of therapeutic alternative and ventilator settings has greatly increased.

### SUMMARY

In accordance with aspects of the present disclosure, a method of issuing an alert for optimizing interaction between a patient and a ventilatory system is provided. The method includes ventilating a patient using a ventilatory system and retrieving one or more of current ventilatory settings, collected ventilatory data, and patient data. The method also includes detecting corruption of interaction between a patient and the ventilatory system, determining one or more root causes of the corruption, and determining and displaying a recommendation for resolving the corruption based on the one or more root causes.

The present disclosure provides new and unique advantages to ventilator treatment over prior ventilator systems. By automatically analyzing collected ventilatory data and patient data from both internal and external sources and generating solutions related to resolving corrupt interaction between a patient and the ventilatory system, the ventilatory system is able to provide a clinician with increased information and decision making capabilities while reducing the amount of time the clinician must spend by the patient's bedside performing assessments and analysis to determine the cause of a problem. By providing the clinician with recommendations in addition to the root cause of a problem, the ventilatory system is able to further assist the clinician and increase the clinician's efficiency by removing a majority of the guesswork and analysis involved in resolving issues related to interaction between the patient and the ventilatory system. This provides an added benefit in short staffing situations by reducing the amount of time a clinician must stay by the patient's bedside to assess the patient and provides an increased confidence to the clinician to resolve such issues in the future. Finally, providing comprehensive solutions for optimizing interaction between patients and ventilatory systems provides clinicians with the ability to provide respiratory therapy to patients in the most efficient manner with little or no interruption, thereby reducing patient recovery time.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described hereinbelow with reference to the accompanying drawings, wherein:

FIG. 1 is a schematic diagram showing a patient receiving respiratory therapy from a ventilatory system comprising a graphical user interface and a respirator, in accordance with an aspect of the present disclosure;

FIG. 2 is a schematic diagram of various subsystems of the graphical user interface of FIG. 1;

FIG. 3 is a flowchart illustrating a method of issuing an alert for optimizing interaction between a patient and a ventilatory system, in accordance with an aspect of the present disclosure; and

FIG. 4 is an illustration showing a graphical user interface displaying an alert identifying a recommended course of action for optimizing interaction between a patient and the ventilatory system, in accordance with an aspect of the present disclosure.

### DETAILED DESCRIPTION

The present disclosure provides a system and method for monitoring and reporting status of a ventilated patient. More specifically, the present disclosure provides a “Waveform Interaction Engine” (WIE) software module that is utilized by a ventilator system to monitor interaction between a patient and a ventilator (patient-ventilator interaction). The WIE may be configured to analyze any combination of collected ventilatory data (e.g., data regarding end-expiratory flow (EEF), data regarding alveolar pressure Pₐ, P_{Peak} data, plateau pressure Pₚₗₐₜ data, volume data, flow trace data, EEP data, time data, etc.), collected physiologic data (e.g., SpO₂ vs. time waveform, expired tidal volume (V_{TE}) vs. time waveform, E_{T}CO₂ vs. tidal volume waveform, etc.), current ventilatory settings, patient data (e.g., Ideal Body Weight (IBW), gender, age, height, patient diagnosis, patient disability, patient post-operative condition, a non-invasive, surrogate waveform representing O₂ (e.g., SpO₂), and a surrogate waveform representing CO₂ (e.g., E_{T}CO₂), etc.), and employ suitable protocols, equations, etc., to detect a corrupted patient-ventilator interaction (e.g., patient-ventilator dyssnchrony) and determine the logical root cause of the corrupted patient-ventilator interaction. Any of the data described above may be collected during inspiration, exhalation, and/or a pause interval between inspiration and exhalation. Further, any of the data described above may be normalized by a predicted or ideal body weight (PBW or IBW) or by any suitable normalization standard to ensure yielding of predicted patient data. A corrupted patient-ventilator interaction may be determined based on certain patient events and/or measurement thresholds (e.g., if measured pressure data is outside of an established upper or lower threshold value). By identifying the logical root cause of a corrupted patient-ventilator interaction in the manner described above, the WIE is able to determine a comprehensive solution to resolving the identified corrupted patient-ventilator interaction. In some embodiments, the WIE may issue a report or an alert to a clinician through a graphical user interface indicating the current status of ventilator-patient interaction whether optimal or suboptimal. In some embodiments, the WIE may alert the clinician through the graphical user interface (e.g., via text and/or graphics) with a recommended course of action to resolve a corrupted ventilator-patient interaction and/or improve ventilator-patient interaction. For example, the recommended course of action may indicate a particular area of interest of the ventilator system to investigate. In some embodiments, the WIE may be configured to automatically provide an update to the current status of the ventilator-patient interaction through the graphical user interface indicating that a corrupted ventilator-patient interaction has been resolved. In some embodiments, the WIE may trigger audible and/or visual alarms through the graphical user interface based on ventilator-patient interaction status, certain patient events, and/or measurement thresholds. For example, a SpO₂ alarm may be configured to activate when the patient's measured SpO₂ is outside of certain threshold values (e.g., upper and lower threshold values). A clinician may configure the threshold values depending on any number of factors such as patient age (e.g., neonate, child, adult), clinical condition (e.g., infarction, cardiac arrest, respiratory illness), clinical history, etc. Any number of alarms may be configured, for example, alarms that activate based on heart rate, temperature, respiration rate, blood pressure, expiratory CO2, etc. Certain alarms may include system alarms configured to activate based on system events and measurements.

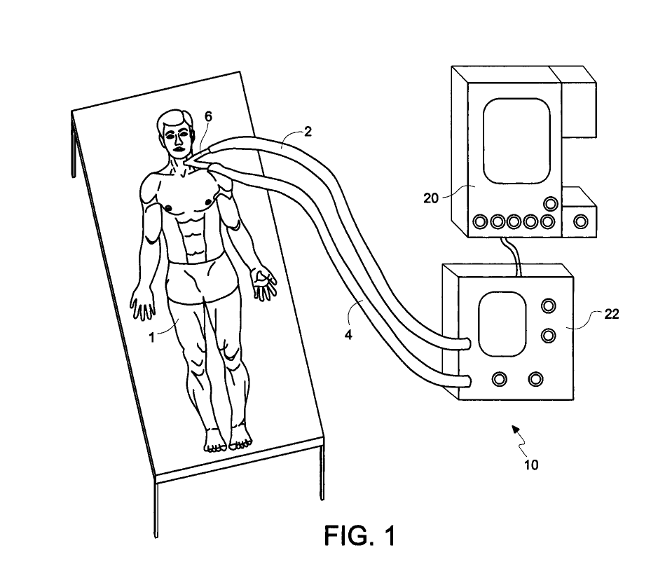

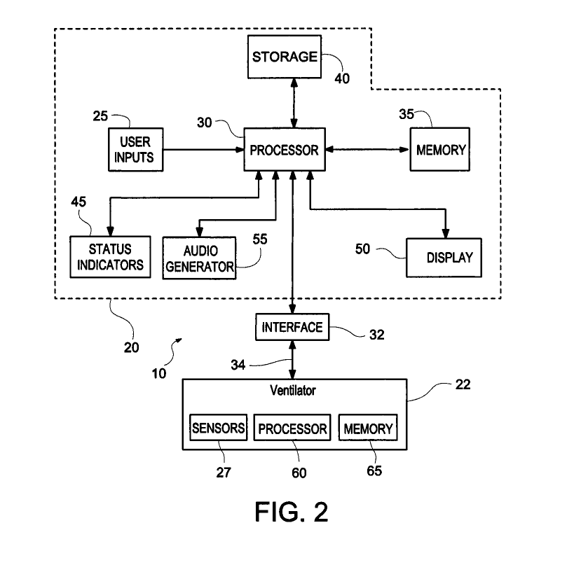

FIG. 1 shows a patient 1 receiving respiratory therapy from a ventilator system 10 having a graphical user interface 20 connected to a ventilator 22. The patient is connected to the ventilator 22 by a patient circuit comprising an inspiratory line 2, and expiratory line 4, and a patient connection tube 6, all connected by a suitable patient connector (not shown). The ventilator 22 includes a data processor 60 (FIG. 2) that controls operation of the ventilator 22.

FIG. 2 depicts the graphical user interface 20 of FIG. 1 in more detail. Generally, the graphical user interface 20 includes user inputs 25, a data processor 30 and memory 35. The memory 35 may be used to store current settings, system status, patient data and ventilatory control software (e.g., the WIE) to be executed by the data processor 30. The data processor 30 may also be connected to a storage device 40, such as battery protected memory, a hard drive, a magnetic tape drive or other storage media for storing patient data and associated ventilator operating parameters. The data processor 30 accepts input received from the user inputs 25 to control the ventilator 22. The graphical user interface 20 may also include status indicators 45, a display 50 for displaying patient data and ventilator settings and an audio generator 55 for providing audible indications of the status of the ventilator system 10.

Ventilator 22 includes memory 65 associated with the data processor 60. Memory 65 includes non-transitory, computer-readable storage media that stores software (e.g., the WIE) that is executed by data processor 60 and which controls the operation of the ventilator 22. In some embodiments, memory 65 includes one or more solid-state storage devices such as flash memory chips. In some embodiments, memory 65 may be mass storage connected to one or more processors (e.g., data processor 60) through a mass storage controller (not shown) and a communications bus (not shown). Although the description of computer-readable media contained herein refers to a solid-state storage, it should be appreciated by those skilled in the art that computer-readable storage media can be any available media that can be accessed by data processor 60. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

The graphical user interface 20 connects to an interface 32 for providing control signals from data processor 30 of the graphical user interface 20 to data processor 60 of the ventilator 22 of the ventilator 22, and also for receiving signals from sensors 27 associated with the ventilator 22 indicative of patient condition and the status of the ventilator 22. The data processor 30 of the graphical user interface 20 may also receive input representative of various clinical parameters indicating clinical condition of the patient 1 and the status of the respiratory therapy from the sensors 27 in the ventilator 22. An electrical cable 34 connects the ventilator 22 to a suitable connector (not shown) of the interface 32.

Ventilator system 10 may include any one or more distributed and/or internal sensors (e.g., sensor 27) communicatively coupled to ventilator 22. Sensors may be placed in any suitable location, e.g., within the ventilatory circuitry or other devices communicatively coupled to the ventilator. For example, sensors may be affixed to the ventilatory tubing or may be imbedded in the tubing itself. In some embodiments, sensors may be provided at or near the lungs (or diaphragm) for detecting a pressure in the lungs.

Sensors may further include pressure transducers that may detect changes in circuit pressure (e.g., electromechanical transducers including piezoelectric, variable capacitance, or strain gauge). Distributed sensors may further include various flow sensors for detecting airflow. For example, some flow sensors may use obstructions to create a pressure decrease corresponding to the flow across the device (e.g., differential pressure pneumotachometers) and other flow sensors may use turbines such that flow may be determined based on the rate of turbine rotation (e.g., turbine flow sensors). Alternatively, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Indeed, any distributed sensory device useful for monitoring changes in measurable parameters during ventilatory treatment may be employed in accordance with embodiments described herein.

Ventilator 22 may further include one or more internal sensors (e.g., sensor 27). Similar to distributed sensors, internal sensors may communicate with various components of ventilator 22, e.g., data processor 60 and any other suitable components and/or modules. Internal sensors may employ any suitable sensory or derivative technique for monitoring one or more parameters associated with the ventilation of a patient. However, the one or more internal sensors may be placed in any suitable internal location, such as, within the ventilatory circuitry or within components or modules of ventilator 22. For example, sensors may be coupled to the inspiratory and/or expiratory modules for detecting changes in, for example, circuit pressure and/or flow. Specifically, internal sensors may include pressure transducers and flow sensors for measuring changes in circuit pressure and airflow. Additionally or alternatively, internal sensors may utilize optical or ultrasound techniques for measuring changes in ventilatory parameters. For example, a patient's expired gases may be monitored by internal sensors to detect physiological changes indicative of the patient's condition and/or treatment. Indeed, internal sensors may employ any suitable mechanism for monitoring parameters of interest in accordance with embodiments disclosed herein.

By way of general overview, a ventilator system may initiate exhalation based on lapse of an inspiratory time setting (T_{I}) or other cycling criteria set by the clinician or derived from ventilator settings (e.g., detecting delivery of prescribed tidal volume delivered (V_{T}) or prescribed inspiratory pressure based on a reference trajectory). Upon initiating the expiratory phase, the ventilator system may allow the patient to exhale by opening an expiratory valve. As such, exhalation is passive, and the direction of airflow, as described above, is governed by the pressure gradient between the patient's lungs (higher pressure) and the ambient surface pressure (lower pressure). Although expiratory flow is passive, it may be regulated by the ventilator system based on the size of the expiratory valve opening.

Expiratory time (T_{E}) is the time from the end of inspiration until the patient triggers for a spontaneously breathing patient. For a non-triggering patient, it is the time from the end of inspiration until the next inspiration based on the set respiratory rate RR. In some cases, however, the time required to return to the functional residual capacity (FRC) or resting capacity of the lungs is longer than provided by T_{E} (e.g., because the patient triggers prior to fully exhaling or the set RR is too high for a non-triggering patient). In some embodiments, various ventilatory settings may be adjusted to better match the time to reach FRC with the time available to reach FRC. For example, decreasing set inspiratory time (T_{I}) to thereby increase the amount of time available to reach FRC. Alternatively, inspiratory pressure may be decreased (decreasing V_{T}), resulting in less time required to reach FRC.

At the point of transition between inspiration and exhalation, the direction of airflow may abruptly change from flowing into the lungs to flowing out of the lungs or vice versa depending on the transition. Stated another way, inspiratory flow may be measurable in the ventilatory circuit until P_{Peak} is reached, at which point flow approximates zero. Thereafter, upon initiation of exhalation, expiratory flow is measurable in the ventilatory circuit until the pressure gradient between the lungs and the body's surface reaches zero (again, resulting in zero flow). However, in some cases, as will be described further herein, expiratory flow may still be positive, i.e., measurable, at the end of exhalation (termed positive end-expiratory flow or positive EEF). In this case, positive EEF is an indication that the pressure gradient has not reached zero or, similarly, that the patient has not completely exhaled. Although a single occurrence of premature inspiration may not warrant concern, repeated detection of positive EEF may be indicative of Auto-Positive End Expiratory Pressure (Auto-PEEP).

As noted above, distributed sensors and internal sensors may collect data regarding various ventilatory parameters. A ventilatory parameter refers to any factor, characteristic, or measurement associated with the ventilation of a patient, whether monitored by the ventilator or by any other device. Sensors may further transmit collected data to the data processor 60. In some embodiments, the data processor 60 may be configured to collect data regarding some ventilatory parameters, to derive data regarding other ventilatory parameters, and to graphically represent collected and derived data to the clinician and/or other modules of the ventilatory system. For example, data regarding end-expiratory flow (EEF), data regarding alveolar pressure Pₐ (e.g., via a breath-hold maneuver), P_{Peak} data, Pₚₗₐₜ data, volume data, flow trace data, EEP data, etc., may be collected, derived, and/or graphically represented by graphical user interface 20. The collected, derived, and/or graphically represented ventilatory data (collected ventilatory data) may be processed and utilized in any combination by the WIE to detect a corrupted patient-ventilator interaction and determine the logical root cause of the corrupted patient-ventilator interaction. The WIE may be configured to issue a report or an alert to a clinician through the graphical user interface 20 indicating the current status of ventilator-patient interaction and/or a recommended course of action to resolve a corrupted ventilator-patient interaction. The report or alert may be provided as a tab, banner, dialog box, text box, pictogram, 3D pictogram, graph, or other suitable type of display on the graphical user interface 20.

In some embodiments, for example, data processor 60 may be configured to monitor inspiratory and expiratory flow. Flow may be measured by any appropriate, internal or distributed device or sensor within the ventilatory system. As described above, flow sensors may be employed by the ventilatory system to detect circuit flow. Data processor 30 may receive flow data monitored by and communicated from data processor 60 and plot the flow data graphically via graphical user interface 20. For example, in some embodiments, flow data may be plotted versus time (flow waveform), versus volume (flow-volume loop), or versus any other suitable parameter as may be useful to a clinician.

In some embodiments, data processor 60 may be configured to monitor pressure. Pressure may be measured by any appropriate, internal or distributed device or sensor within the ventilatory system. For example, pressure may be monitored by proximal electromechanical transducers connected near the airway opening. Alternatively, pressure may be monitored distally, at or near the lungs and/or diaphragm of the patient. Data processor 30 may receive pressure data monitored by and communicated from data processor 60 and plot the pressure data graphically via graphical user interface 20. For example, in some embodiments, pressure data may be plotted versus time (pressure waveform), versus volume (pressure-volume loop or PV loop), or versus any other suitable parameter as may be useful to a clinician.

In some embodiments, PV loops may provide useful clinical and diagnostic information to clinicians regarding the respiratory resistance or compliance of a patient. Specifically, upon comparing PV loops from successive breaths, an increase in resistance may be detected when successive PV loops shorten and widen over time. That is, at constant pressure, less volume is delivered to the lungs when resistance is increasing, resulting in a shorter, wider PV loop. If a lung has a resistance R_{L} and a compliance C_{L}, but is not constrained by a filling time, then the final lung volume (V_{L})=P_{L}*C_{L}. If resistance R_{L} of the lung is considered in calculating the final lung volume V_{L}, then the final lung volume V_{L}=P_{L}*C_{L}[1−exp(−t/R_{L}*C_{L})], where R_{L}*C_{L} is the time constant and t is time. If inspiratory time is greater than three time constants relative to time t, then the lung will fill regardless of the value of t. If inspiratory time is less than three time constants relative to time t, the final lung volume V_{L} will be less than the equilibrium volume.

In some embodiments, a PV loop may provide a visual representation, in the area between the inspiratory plot of pressure vs. volume and the expiratory plot of pressure vs. volume, which is indicative of respiratory compliance. Further, PV loops may be compared to one another to determine whether compliance has changed. Additionally or alternatively, optimal compliance may be determined.

In some embodiments, data processor 60 may be configured to derive lung volume delivered to the patient during inspiration as well as the volume of gas in a patient's lung that produced a given exhalation. For example, as described above, during volume ventilation, a prescribed volume V_{T} may be set for delivery to the patient. The actual volume delivered may be derived by monitoring the inspiratory flow over time (i.e., V=F*t) at a point closest to the patient where gas exited the ventilator system 10 and passed into the patient. Stated differently, integration of flow over time will yield volume. In some embodiments, delivered flow is measured by sensors (e.g., sensors 27) disposed within the ventilator system 10 and volume of the ventilator system 10 is calculated by a suitable algorithm executed by the data processor 60 and subtracted from the total delivered volume to yield the volume delivered to the patient. In some embodiments, V_{T} is completely delivered upon reaching T_{I}. Similarly, the expiratory flow may be monitored such that expired tidal volume (V_{TE}) may be derived. That is, under ordinary conditions, upon reaching the T_{E}, the prescribed V_{T} delivered should be completely exhaled and FRC should be reached. However, under some conditions T_{E} is inadequate for complete exhalation and FRC is not reached. Data processor 30 may 60 may be further configured to plot derived volume data graphically via any suitable means. For example, in some embodiments, volume data may be plotted versus time (volume waveform), versus flow (flow-volume loop or FV loop), or versus any other suitable parameter as may be useful to a clinician.

The root cause of corrupt patient-ventilator interaction and the best course of action to eliminate the root cause may be difficult for a clinician to determine. As described above, the WIE may be configured to determine the root cause of corrupted patient-ventilator interaction based on, for example, current ventilatory settings, collected ventilatory data, patient data, and any suitable protocols, equations, etc. For example, the WIE may first calculate a root cause by analyzing one or more waveforms of ventilatory data (e.g., flow waveform, pressure waveform, etc.) and/or waveforms of physiologic data (e.g., SpO2 versus time, V_{TE} versus time, E_{T}CO₂ versus tidal volume, etc.). Thereafter, the WIE may determine a recommended course of action or actions for resolving corrupted patient-ventilator interaction for a particular ventilated patient and alert a clinician via the graphical user interface 20 accordingly. The recommended course of action may take into account how various ventilatory parameters, patient condition, and/or patient treatment may be impacted if the recommended course of action is taken, which may not be readily apparent to a clinician. In some embodiments, the alert may display the recommended course of action and the projected impact on the patient if the recommended course of action is ignored.

In some embodiments, a root cause of corrupt patient-ventilator interaction may be determined based on analysis of a plurality of waveforms, a plurality of waveform types, a plurality of settings, a plurality of patient data, or any combination thereof, and how changes or adjustments of one may impact another. For instance, a breach of an established upper or lower threshold value for one waveform (e.g., pressure waveform) may impact another waveform (e.g., flow waveform) or one corrupt condition may compound another. As such, the WIE may be configured to consider all or any combination of current ventilatory settings, collected ventilatory data, patient data, protocols, equations, etc. to determine the root cause and alert the clinician with a recommended course of action to eliminate the root cause and optimize patient-ventilator interaction.

The alert may be provided as a tab, banner, dialog box, text box, pictogram, graph, or other suitable type of display on the graphical user interface 20. Further, the alert may be provided along a border of the graphical user interface, near an alarm display or bar, or in any other suitable location. A shape and size of the alert may further be optimized for easy viewing with minimal interference to other ventilatory displays. The alert may be further configured with a combination of icons and text such that the clinician may readily identify the recommended course of action.

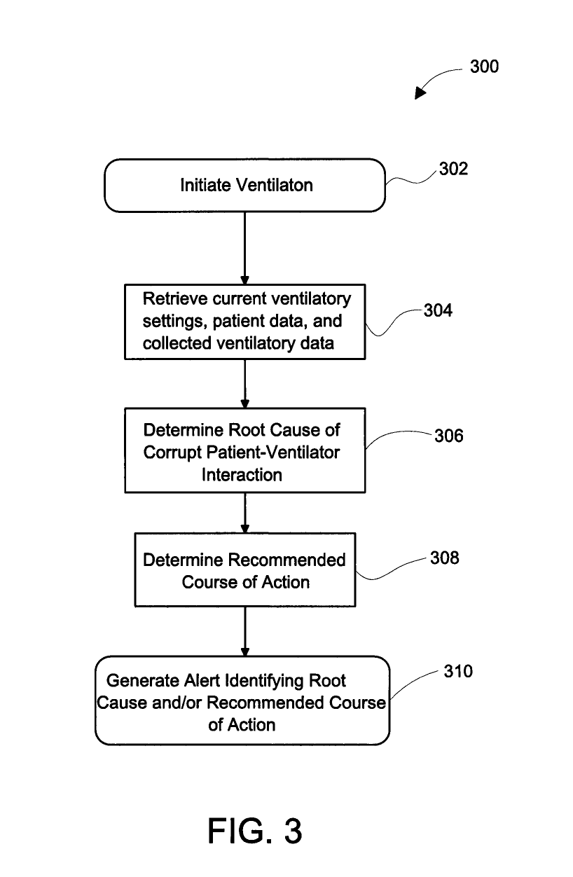

FIG. 3 is a flow chart illustrating an embodiment of a method for issuing an alert indicating a recommended course of action to optimize patient-ventilator interaction.

The particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

Method 300 begins with an initiate ventilation operation 302. Initiate ventilation operation 302 may further include various additional operations. For example, initiate ventilation operation 302 may include receiving one or more ventilatory settings associated with ventilation of a patient. As such, the ventilatory settings and/or input received may include, for example, an inspiratory pressure (or target inspiratory pressure), a tidal volume (V_{T}), a respiratory rate (RR), etc. In addition, during initiate ventilation operation 302, patient data may be received. Patient data may refer to any data particular to a patient, for example, a predicted or ideal body weight (PBW or IBW), a patient diagnosis, a patient age, a patient disability, a patient post-operative condition, etc. The patient data may be manually entered or retrieved from a patient's electronic medical record (EMR), which may be stored on a hospital server or other network data storage system to which the ventilator system 10 may be connected. A patient diagnosis may include ARDS, COPD, emphysema, asthma, etc. Upon initiating ventilation, the WIE may further monitor ventilatory parameters and collect ventilatory data.

At retrieve operation 304, the WIE may retrieve current ventilatory settings, patient data (including a patient diagnosis) stored in memory 65, and collected ventilatory data from the data processor 60. According to embodiments, the WIE may further retrieve any other suitable data or information as needed for monitoring patient-ventilator interaction and/or determining a root cause of corrupt patient-ventilator interaction.

At determine root cause operation 306, the WIE may determine a root cause of corrupt patient-ventilator interaction based on, without limitation, current ventilatory settings, patient data, collected ventilatory data, and/or any other useful information in the form of waveforms, equations, conversion tables, etc., any one or more of which may be combined and analyzed to determine the root cause. For example, the WIE may analyze a PEEP setting of the ventilator 22, measured end-expiratory pressure (EEP), an expiratory flow waveform, and an end-expiratory pressure waveform to determine if Auto-PEEP is likely. A non-zero expiratory flow waveform and a pressure at the patient-ventilator coupling (e.g., at a wye connection disposed along a tubing system coupling the patient with the ventilator) above a set PEEP threshold value may be indicative of Auto-PEEP.

At determine recommended course of action operation 308, the WIE may determine a recommended course of action for a clinician to take to eliminate the root cause of corrupt patient-ventilator interaction. In some embodiments, for example, the WIE may determine the recommended course of action for a particular ventilated patient. That is, where the patient exhibits normal resistance and/or compliance and where the determined root cause is an unexpected increase in E_{T}CO₂, the WIE may recommend increasing pressure and/or V_{T} (e.g., by comparison with institutional protocol thresholds, manufacturer protocol thresholds, clinician protocol thresholds, prescribed patient ranges, etc., stored in memory 65). The WIE may compare differential values of flow, volume, and pressure with set absolute or ideal body weight based values to determine a recommended course of action. For example, the WIE may determine that, at the time the patient triggered the next breath, the end expiratory pressure (EEP) was 4 cmH₂O above a set PEEP and the end-expiratory flow was −10 L/min. The combination of these determinations may suggest Auto-PEEP. The WIE may also compare an inspiratory time setting (T_{I}) and an expiratory time setting (T_{E}) to determine if the respiratory rate (RR) is too high. An analysis of an expiratory flow trace may lead to a conclusion that obstructive exhalation is present.

At generate alert operation 310, the WIE may generate an alert identifying the recommended course of action to resolve a fault condition and/or the root cause of the fault condition. For example, the WIE may generate an alert identifying the recommended course of action and the root cause in text form via the graphical user interface 20. Additionally, the determinations and conclusions described above with respect to operation 308 may be presented to the clinician via the graphical user interface 20 for verification and be accompanied by suggestions to improve patient management. For example, suggestions to improve patient management may include, without limitation, a suggestion to reduce respiratory rate if in a controlled ventilation environment, a suggestion to add a set PEEP to maintain open airways during exhalation, a suggestion to slightly increase the prescribed volume (V_{T}) to enhance expiratory pressure gradient, and/or a suggestion to decrease the inspiratory time (T_{I}) setting and increase the expiratory time (T_{E}) setting.

In some embodiments, a clinician may interact with the graphical user interface 20 to instruct the WIE to perform an action (e.g., the recommended course of action) to resolve the root cause of corrupt patient-ventilator interaction. After the WIE performs the action to resolve the root cause of the corruption, the WIE may indicate, via the graphical user interface 20, that the action has been performed. In some embodiments, the WIE may determine and verify, via the graphical user interface 20, that the root cause of the corruption has been resolved.

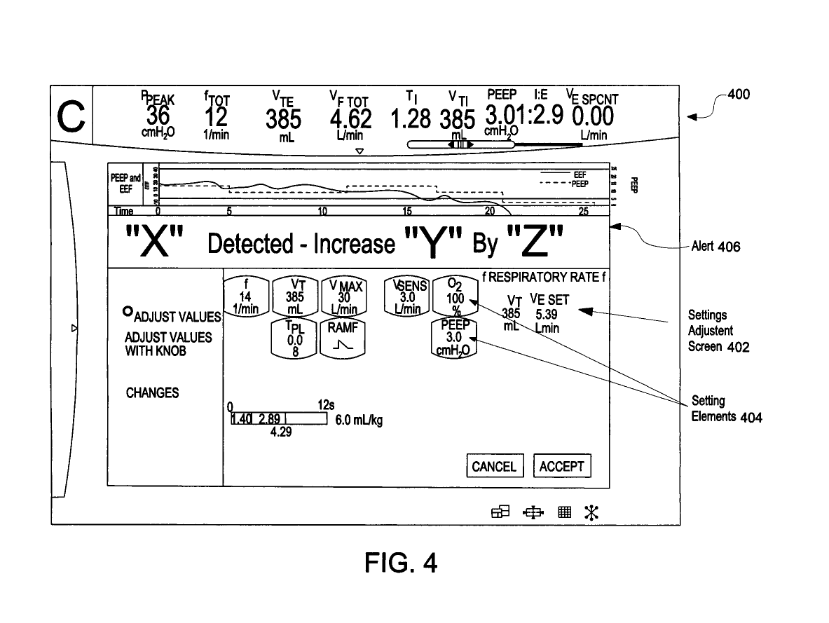

FIG. 4 is an illustration of an embodiment of a graphical user interface displaying an alert identifying a recommended course of action for resolving corrupt patient-ventilator interaction.

Graphical user interface 400 may display a settings adjustment screen 402. The settings adjustment screen 402 may be accessed via a settings icon or other control. The settings adjustment screen 402 may provide one or more settings elements 404. The one or more settings elements 404 may display actual settings values corresponding to current ventilatory settings. Settings may be adjusted via direct input into a settings input field, via use of a scroll wheel, thumbwheel, knob, mouse, or scroll bar for adjusting settings up and down, touchscreen, or via any other suitable device.

An alert may be displayed on the graphical user interface 400, e.g., alert 406. As described above, alert 406 may be displayed as a tab, banner, dialog box, or other suitable type of display, along a border of the graphical user interface, near an alarm display or bar, or in any other suitable location. As illustrated, alert 406 is displayed as a bar above and adjacent to the settings adjustment screen 402. However, alert 406 may be located along any border region of the graphical user interface 400 (e.g., top, bottom, or side borders) (not shown) or in any other suitable location. Further, alert 406 may be partially transparent (not shown) such that ventilatory displays and data may be at least partially visible behind the alert. Alert 406 may further provide a root cause of a fault condition and/or a recommended course of action for resolving the fault condition based on the root cause. In some embodiments, alert 406 may be configured to simply display a report indicating the current status of ventilator-patient interaction.

The disclosed data, graphics, and alert illustrated in graphical user interface 400 may be arranged in any suitable order or configuration such that information and recommendations may be communicated to the clinician in an efficient and orderly manner. The disclosed data, graphics, and alert are not to be understood as an exclusive array, as any number of similar suitable elements may be displayed for the clinician within the spirit of the present disclosure. Further, the disclosed data, graphics, and alert are not to be understood as a necessary array, as any number of the disclosed elements may be appropriately replaced by other suitable elements without departing from the spirit of the present disclosure. The illustrated embodiment of the graphical user interface 400 is provided as an example only, including potentially useful information and recommendations that may be provided to the clinician to facilitate communication of a course of action to resolve a fault condition in an orderly and informative way, as described herein.

While the graphical user interface 400 is depicted and described as appearing on a display (e.g., display 50) of a ventilator system (e.g., ventilator system 10), it should be understood that the use interface 400 may appear on any device connected to a hospital network to which the ventilator system 10 is also connected (e.g., via LAN, WAN, WiLAN, etc.). Accordingly, the graphical user interface 400 may be presented on a central nurse's station computer, on a tablet or smartphone carried by a clinician, or any other device having access to the network on which the ventilator system 10 resides. The generation of the user interface 400 may require the use of one or more servers to store and present data to the various devices including a data collection server, an application server, and a web server whose functions may be performed by separate devices or a single device.

What is claimed is:

1. A method of issuing an alert for optimizing interaction between a patient and a ventilatory system, comprising:
ventilating a patient using a ventilatory system;
retrieving at least one of current ventilatory settings, collected ventilatory data, and patient data;
detecting corruption of interaction between a patient and the ventilatory system;
determining at least one root cause of the corruption; and
determining and displaying a recommendation for resolving the corruption based on the at least one root cause.

2. The method according to claim 1, further comprising displaying the at least one root cause.

3. The method according to claim 1, further comprising displaying a report indicating a status of the interaction between a patient and the ventilatory system.

4. The method according to claim 1, further comprising issuing an alert associated with the detected corruption of interaction between a patient and the ventilatory system.

5. The method according to claim 1, wherein the patient data comprises at least one of a patient diagnosis and a patient disability.

6. The method according to claim 1, further comprising normalizing the patient data based on at least one normalization standard.

7. The method according to claim 1, further comprising displaying at least a portion of the collected ventilatory data to graphically represent the root cause of the corruption.

8. The method according to claim 1, wherein the collected ventilatory data comprises at least one of end-expiratory flow data, alveolar pressure data, flow trace data, and end-expiratory pressure data.

9. The method according to claim 1, wherein the collected ventilatory data comprises at least one of flow data, pressure data, volume data, and time data.

10. The method according to claim 1, wherein the collected ventilatory data may be collected during at least one of inspiration, exhalation, and a pause interval between inspiration and exhalation.

11. The method according to claim 1, wherein the patient data comprises at least one of a non-invasive, surrogate waveform representing O₂, and a surrogate waveform representing CO₂.

12. The method according to claim 1, wherein determining at least one root cause of the corruption is based on at least one of the current ventilatory settings, the collected ventilatory data, and the patient data.

13. The method according to claim 1, further comprising representing the recommendation as at least one of a tab, a banner, a dialog box, a text box, a pictogram, a 3D pictogram, and a graph.

14. A ventilatory system for issuing an alert for optimizing interaction between a patient and a ventilatory system, comprising:
a graphical user interface;
a ventilator configured to ventilate a patient;
at least one data processor; and
at least one memory configured to communicate with the at least one data processor, the at least one memory storing instructions executable by the at least one processor to perform a method comprising:
retrieving at least one of current ventilatory settings, collected ventilatory data, and patient data;
detecting corruption of interaction between a patient and the ventilatory system;
determining at least one root cause of the corruption;
determining a recommendation for resolving the corruption based on the at least one root cause; and
displaying the recommendation via the graphical user interface.

15. The ventilatory system according to claim 14, further comprising displaying the at least one root cause via the graphical user interface.

16. The ventilatory system according to claim 14, further comprising displaying a report indicating a status of the interaction between a patient and the ventilatory system via the graphical user interface.

17. The ventilatory system according to claim 14, further comprising:
performing an action to resolve the root cause of the corruption based on input from a clinician; and
indicating, via the graphical user interface, that the action has been performed.

18. The ventilatory system according to claim 17, further comprising verifying that the root cause of the corruption has been resolved via the graphical user interface.

19. A ventilator, comprising:
at least one data processor;
at least one memory configured to communicate with the at least one data processor, the at least one data processor configured to retrieve at least one of current ventilatory settings, collected ventilatory data, and patient data to detect corruption of interaction between a patient and the ventilator, wherein the at least one processor determines a recommendation for resolving the corruption based on at least one root cause of the corruption, and
a graphical user interface configured to display an alert including the recommendation for optimizing interaction between a patient and the ventilator.

20. The ventilator of claim 16, wherein the graphic user interface further displays the at least one root cause.
